(19) United States
(12) Patent Application Publication
(10) Pub. No.: US 2009/0024410 A1
(43) Pub. Date: Jan. 22, 2009

(54) VERTICAL CONTENT SEARCHING METHOD AND APPARATUS

(76) Inventors: David Burgess, Menlo Park, CA (US); Shyam Kapur, Sunnyvale, CA (US)

Correspondence Address: YAHOO! INC.;C/O DREIER LLP, 499 PARK AVENUE, NEW YORK, NY, 10022, US

(21) Appl. No.: 11/778,446

(22) Filed: Jul. 16, 2007

(51) Int. Cl. G06F 17/30; G06Q 50/00

(52) U.S. Cl. 705/2; 707/100; 707/3

(57) ABSTRACT

The present invention provides for conducting a vertical search, wherein a vertical search is a content search in a defined or otherwise limited domain. The method and apparatus includes receiving a search request from a user, where the user has a user profile with user profile data associated therewith. The method and apparatus includes defining a search domain based on the search request and the user profile data. Thereby, the method and apparatus includes conducting a content search within the search domain, as the search domain is defined in part by the user-profile information and thereby provides a much more concise search result. Additionally, the method and apparatus may perform additional functions based on the aggregation of data across numerous users having related profile data.

### COPYRIGHT NOTICE

A portion of the disclosure of this patent document includes material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF INVENTION

The present invention relates to content searching. More specifically, the present invention is directed to facilitating a search that returns more relevant results to a search query based on user profiles and prior searches.

### BACKGROUND OF THE INVENTION

The goal of a search engine is to find the most relevant information. Popular search engines such as Yahoo!, Google, and MSN, have relied on traditional horizontal approaches to searching. Horizontal search engines are usually characterized by a lack of a hierarchal structure. Horizontal search engines rely on search crawlers that typically index almost everything over the Internet or a network. The results they produce span a very broad range of categories. However, users frequently desire search results from a narrower range of categories.

Since search queries tend to be short, one technique would require the search query to be more complex. Some search systems allow users to input complex logic queries in order to get better results. This approach is undesirable because the required search format in such systems is no longer intuitive. Some users would therefore not take the time to learn to use a system and avoid it altogether. Also it takes more time and effort for one familiar with the particular search format to craft a proper search statement.

Existing systems also fail to account for benefits that can be achieved with data related to the search and the search requesting entity. Many improvements in searching technology relate to techniques for improving the accuracy of the search engine. For example, existing systems do not coordinate searching requests with available information about the user making such request. Rather, existing systems tend to be response-oriented, providing search results directly in response to search requests.

Hence, there is a need for a robust, practical approach to narrow search results to user desired topic domains while allowing search queries to remain simple and brief.

### SUMMARY OF THE INVENTION

The present invention provides for conducting a vertical search, wherein a vertical search is a content search in a defined or otherwise limited domain. The method and apparatus includes receiving a search request from a user, where the user has a user profile with user profile data associated therewith. The method and apparatus includes defining a search domain based on the search request and the user profile data. Thereby, the method and apparatus includes conducting a content search within the search domain, as the search domain is defined in part by the user-profile information and thereby provides more relevant search results. Additionally, the method and apparatus may perform additional functions based on the aggregation of data across numerous users having related profile data.

### BRIEF DESCRIPTION OF THE DRAWINGS

The invention is illustrated in the figures of the accompanying drawings which are meant to be exemplary and not limiting, in which like references are intended to refer to like or corresponding parts throughout, and in which:

FIG. 1 is a block diagram illustrating a system for conducting a search of a query according to one embodiment of the present invention;

FIG. 2 is a flow diagram illustrating a method for producing search results in response to a user query according to one embodiment of the present invention;

FIG. 3 is block diagram illustrating another embodiment of a system for conducting a search of a query according;

FIG. 4 is a flow diagram illustrating a method for knowledge based search in an embodiment relating to vehicle information; and

FIG. 5 is a flow diagram illustrating a method for knowledge based search in an embodiment relating to medical information.

### DETAILED DESCRIPTION OF THE INVENTION

In the following description of embodiments of the invention, reference is made to the accompanying drawings that form a part hereof and in which is shown by way of illustration a number of specific embodiments in which the invention may be practiced. It is to be understood that other embodiments may be utilized and structural changes may be made without departing from the scope of the present invention.

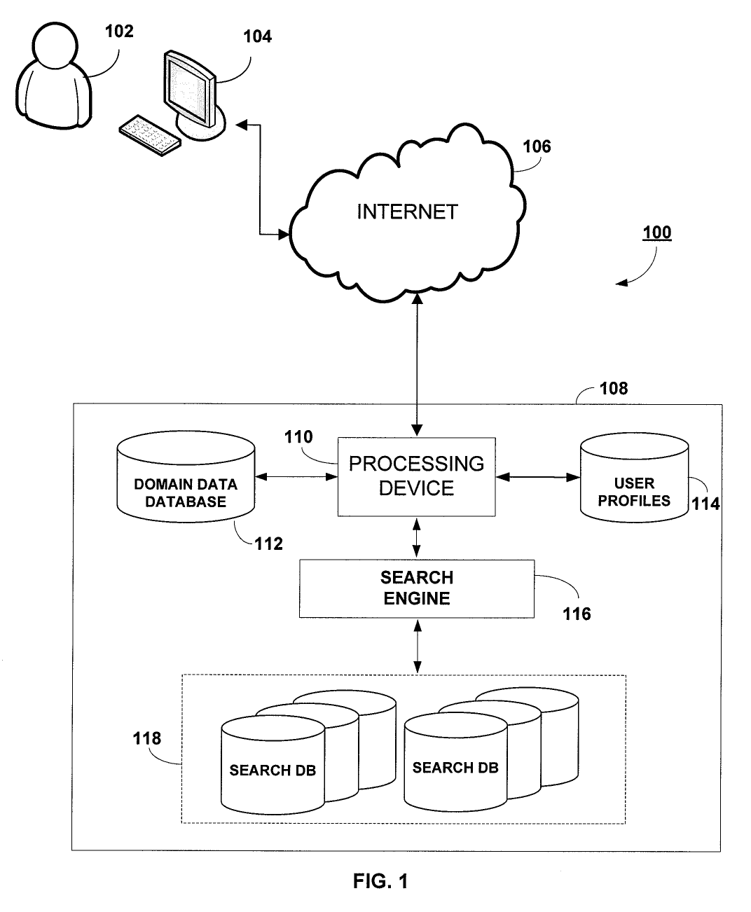

FIG. 1 presents a block diagram illustrating an embodiment of a system 100 allowing for vertical searching. The system 100 includes a user 102 having a computer 104 connected to the Internet 106. The system 100 also includes a processing system 108, which includes a processing device 110, a user profile database 112, a domain data database 114, a search engine 116 and a plurality of search databases 118.

The user 102, through the computer 104 in communication with the system 108 via the Internet, may conduct a vertical search. In other embodiments, other devices that may be used besides a computer include, but are not limited to, personal digital assistants (PDAs), cellphones, and client devices that connect to suitable output devices, such as a television or monitor.

According to an embodiment of FIG. 1, the system 108 may consist of a web server providing a web portal accessed through a defined universal resource location (URL). In the system 108, the processing device 110 may be one or more processing elements operative to perform processing operations in response to executable instructions. The instructions may be stored on and provided from any suitable memory device (not explicitly shown).

Similarly, the search engine 116 may also include operations performed by one or more processing devices in response to executable instructions. The search engine 116 may perform search operations in accordance with known searching techniques. It is also recognized that the search engine 116 may be disposed directly within the processing device 110, whereby search engine executable instructions are executable by the same processing elements, but have been illustrated as separate components in FIG. 1 for clarity purposes only.

The system 108 is also illustrated as including numerous databases, including the domain data database 112, user profile database 114 and the search databases 118. These databases 112, 114 and 118 may be one or more storage devices that can be locally or remotely disposed relative to the processing device 110 and the search engine 116. The domain data database 112 includes domain data, where the domain data provides an indication of specialized or otherwise refined search context, as described in further detail below as the search domain. The user profile database 114 includes user profile data, where, as described in further detail below, includes data associated with user profiles of the users (including the profile of user 102). Additionally, the search database 118 represents any number of suitable databases of information or content that can be searched by the search engine 116, where the direction of the search and/or the content in the search databases may be refined by the search domain.

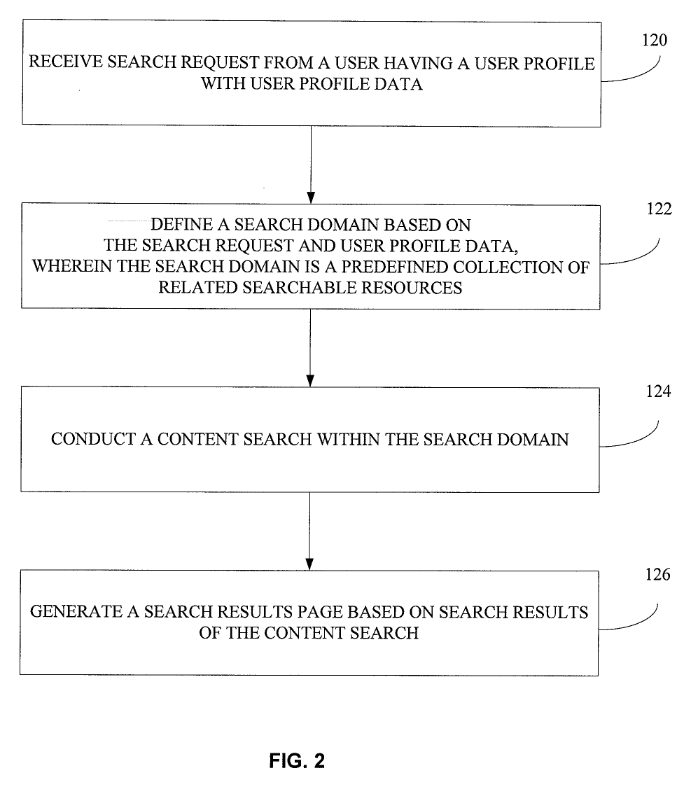

With respect to the system 100 of FIG. 1, FIG. 2 illustrates a flowchart of the steps of one embodiment of a vertical searching method, as may be performed by the processing device 110 within the device 108. A first step, step 120, is the receipt of a search request from a user having a user profile. The user 102 may provide the search request, wherein the profile database 114 may store the user profile data therein. The user profile data may be data entered by the user on the search engine web portal or could be information made available from another source. For example, if the user has an electronic mail account or an online discussion board registration from the same portal as the search engine, the user profile data may include information entered for these additional purposes. In another example, larger commercial databases may be cross-referenced by the user profile information to provide this user profile data, such as an insurance database which could include information on a person's automobile, by way of example.

The search request may be consistent with standard search requests, such as the user 102 entering a search term on the user's computer 104, and the computer 104 transmitting this search request to the processing device 110 via the Internet 106.

The next step, step 122, is defining a search domain based on the search request and the user profile data. The search domain defines a collection of related searchable resources that are related to the corresponding domain or topic. For example, if the search request includes terms that relate to an automobile, the processing device 110 may recognize the search term and then define search domain accordingly. One possible technique may include a look-up table or other types of recognition algorithms and a possible matching technique to correspond the search term to predefined search domains.

Consistent with the search domains, as predefined and stored in the domain database 112, the corresponding domains may be attributed or associated in the search databases 118. Thereby, the next step, step 124, is conducting a content search within the search domain. This content search may include searching techniques that filter all results found on the internet that are outside of the predefined domain, for example a pre-determined list of acceptable root URLs. In another technique, the search domain may include a pre-determined list of root URLs through which the searching may be conducted. In another embodiment the search content may be imported from domain-specific data sources (e.g. a company's intranet or 3^{rd} party databases).

Step 124 conducts the vertical search. The constraints of the vertical search are defined by the search domain in step 122, where the search domain is defined by both the search request (which may include the domain and content of the search web-page) and the user profile data. Whereas prior searching techniques conducted full horizontal searches across Internet-based resources, the device 108 conducts the vertical search on the specified domain, where this specified domain is determined based on the user profile and the search itself.

Consistent with conducting a search, the next step in this embodiment includes generating a search results page based on search results of the content search, step 126. The search results page may thereupon be provided back to the user 102 via the Internet 106 for display on the computer 104.

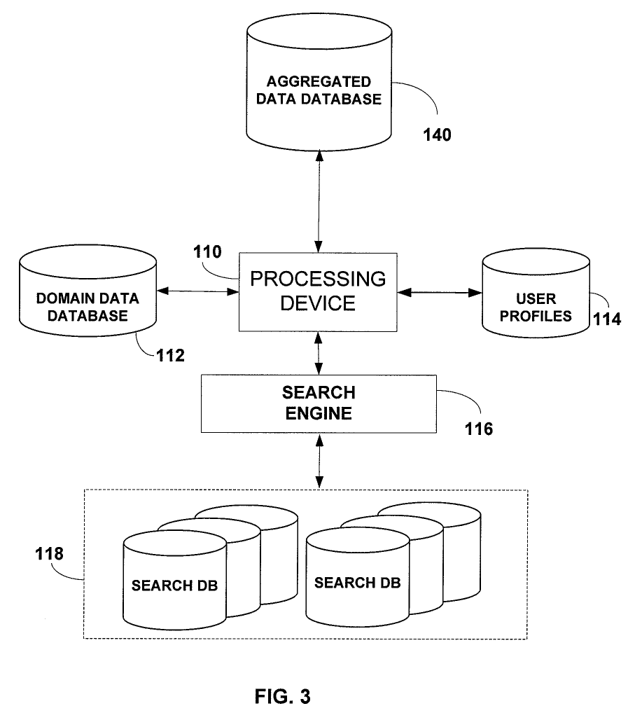

FIG. 3 illustrates another embodiment, wherein the processing device 110 is coupled to an aggregated data database 140. The processing device 110 includes the previously-described elements of the domain data database 112, user profile database 114, search engine 116 and search database 118. The aggregated data database 140 may be one or more data storage devices storing data in either a central or distributed data storage manner.

The processing device 110 is operative to perform further processing operations using aggregated data stored in the aggregated database 140. The processing device may further perform additional processing operations to aggregate the data for storage in the database 140.

The aggregate data may include the search data, such as the search terms used in the search as well as the associated domains. The aggregated data may include other available data, for example, data on user selections on the search results. The aggregate data may also include user profile data, where applicable. The processing device 110 may access any number of user profiles and be able to aggregate data across different user profiles.

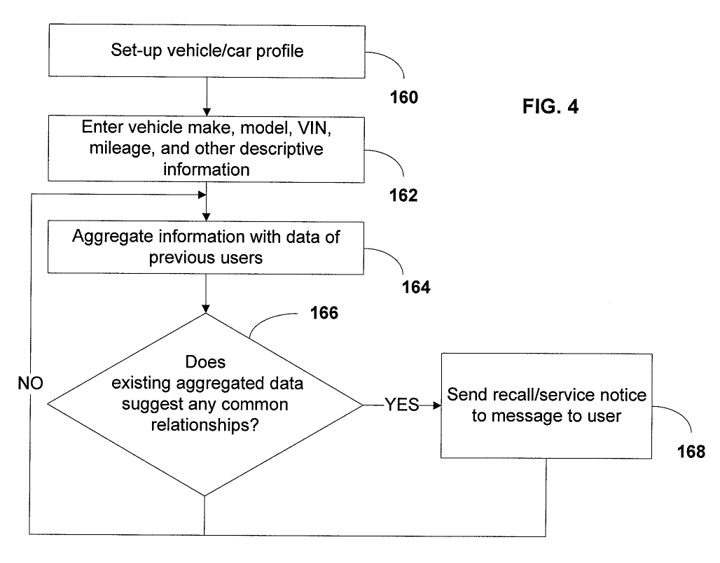

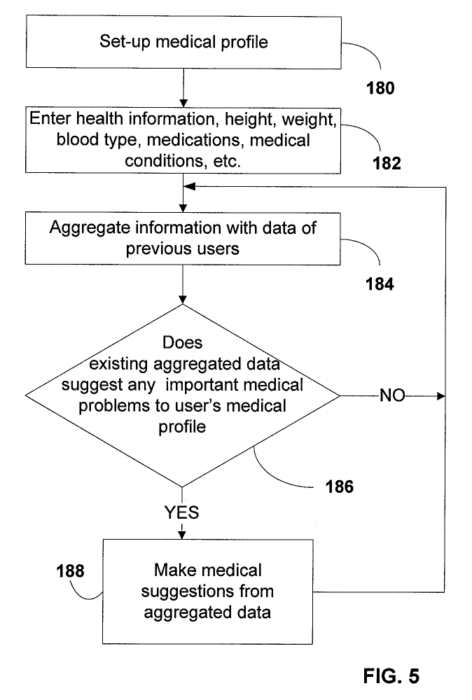

The aggregation of the data may include processing operations or techniques to filter or otherwise manipulate the data. For example, one technique may be determining relationships between different search domains, search terms and user profiles. This aggregated data may thereupon be used for additional purposes, such as pro-active steps based on pattern recognition. FIG. 4 illustrates the steps of a flowchart of a sample embodiment of vertical search and subsequent data aggregation operations relative to vehicle information and FIG. 5 illustrates the steps of a flowchart of one embodiment of a vertical search and subsequent data aggregation operations relative to medical information.

FIG. 4 shows an embodiment of the present invention where a person has set-up a profile with specific interests. In the first step, a person in this embodiment sets-up a profile for his vehicle, step 160. For example, through a website, a user may indicate he owns a car and may be prompted to set up a profile including not only the user's personal information, but also information on the vehicle itself. This may be done at a section of the content provider's site that provides specific information for car owners.

The person enters information specific to his vehicle, including make, model, VIN, mileage, etc, step 162. It is recognized that the profile information including vehicle information may also be made available from additional sources, such as for example a State Vehicle Registration database, an insurance database, a Vehicle Identification Number database, or any other suitable location. The information may be entered through a web form or other types of input methods.

According to some embodiments, a user interface may be provided through which a user may provide additional symptomatic or descriptive information regarding one or more issues or problems that the user is experiencing. In response to the additional symptomatic or descriptive information that the user provides, business logic at the client or server may pose additional questions to narrow a search. For example, when providing information regarding a vehicle, the system may present the user with a graphical representation of the make and model of the car the user owns, allowing the user to graphically indicate issues with the vehicle, in response to which the system may pose additional questions.

The profile input received by a content provider site, for example, then may be aggregated with previous user's profiles, step 164. The aggregation step may include processing the data to recognize commonalities therebetween, for example finding all users having the same make and model of a particular vehicle.

The aggregated data may also include search engine information, as well as the user profile data. For example, the search engine information, which may be stored in the user profile data, may indicate that a number of users have conducted different vertical searches using similar search terms or have selected similar search result links. Therefore, step 166 includes a determination of whether the aggregated data suggests a common feature. For example, it may be discovered that user's profile matches well with prior users who had a similar car. It may be discovered that these prior users recently needed to have their cars serviced. Or in other instances, it may be found that users with the nearly identical cars were recently performing searches asking for information about a product recall. By way of more specific example, the aggregated data may recognize that users having the common vehicular profile data relating to a “Ford Explorer”® were conducting searches on the terms “tire,” “recall,” and “blowout.”

If the inquiry to step 166 is in the affirmative, the system may thereupon perform an action based on this aggregated data determination, step 168. For example, one step may be issue a notification, such as via email, to all users having the vehicle information in their profile. Another example may be to notify the corresponding vehicle manufacturer about this information so the manufacturer may decide to issue a recall. Another example may be to notify an insurance company or a state or Federal agency about this aggregated data. There are additional actions that may be taken using this mined aggregated data, as recognized by one skilled in the art.

In this flowchart, it is recognized that one or more vertical searches can be conducted between steps 162 and 164. The vertical searches, as described in detail above, provide additional information used for the data aggregation operations. Therefore, in the event the inquiry of step 166 is in the negative, the method may revert back to step between steps 162 and 164 whereby additional vertical searches may be conducted.

FIG. 5 illustrates a flow diagram of another embodiment according to the present invention. The user sets up a medical profile, step 180. A person may enter his personal health information, step 182, or in another embodiment, the information may be loaded from another source, such as a health-care provider database, for instance. Unlike the car profile, this profile may be set up so that the health information cannot be linked to an identifiable person for privacy reasons.

According to some embodiments, a user interface may be provided through which a user may provide additional symptomatic or descriptive information regarding one or more issues or problems that the user is experiencing. In response to the additional symptomatic or descriptive information that the user provides, business logic at the client or server may pose additional questions to narrow a search. For example, when providing information regarding health information, the system may present the user with a graphical representation of the human form, allowing the user to graphically indicate issues with the on his or her body, in response to which the system may pose additional questions.

The medical data may be aggregated with existing user's medical information to create a rich database of medical information, step 184. Between steps 182 and 184, users logged on to the system or otherwise recognized by the search engine may conduct vertical searching in accordance with the above-described technique. The next step, step 186, is to examine the aggregate of user profile medical data as well as search data to determine if any relations exist. Another option may be to estimate if the person is at risk to any medical problems, for example if the user's profile indicates a specific body mass index (BMI) and individuals having similar profiles have been conducting searches relating to “hypertension”.

If a relation is found, search results or messages or other types of notifications may be sent out to alert the user to the possible dangers given his medical profile, 188. The message may include information about the particular medical situation as well as possible treatment. The message may suggest physicians or other resources that may describe the medical condition. If no relationships are found in the aggregated data, the method may revert back to additional vertical searching, prior to step 184.

This embodiment may include additional features or functions relating to or providing for diagnosis. For example, the user profile data may be cross-referenced against or used as a cross-referencing agent for any number of suitable content databases. By way of example, one content database may be an audio database of doctor/patient interactions with specific keyword or term recognition operations, where patient-identifying information can be removed for privacy protection concerns. Another example may be photographs or visual information used for comparison purposes, e.g. comparing types of rashes.

FIGS. 1 through 5 are conceptual illustrations allowing for an explanation of the present invention. It should be understood that various aspects of the embodiments of the present invention could be implemented in hardware, firmware, software, or combinations thereof. In such embodiments, the various components and/or steps would be implemented in hardware, firmware, and/or software to perform the functions of the present invention. That is, the same piece of hardware, firmware, or module of software could perform one or more of the illustrated blocks (e.g., components or steps).

In software implementations, computer software (e.g., programs or other instructions) and/or data is stored on a machine readable medium as part of a computer program product, and is loaded into a computer system or other device or machine via a removable storage drive, hard drive, or communications interface. Computer programs (also called computer control logic or computer readable program code) are stored in a main and/or secondary memory, and executed by one or more processors (controllers, or the like) to cause the one or more processors to perform the functions of the invention as described herein. In this document, the terms “machine readable medium,” “computer program medium” and “computer usable medium” are used to generally refer to media such as a random access memory (RAM); a read only memory (ROM); a removable storage unit (e.g., a magnetic or optical disc, flash memory device, or the like); a hard disk; electronic, electromagnetic, optical, acoustical, or other form of propagated signals (e.g., carrier waves, infrared signals, digital signals, etc.); or the like.

Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A vertical searching method comprising:
receiving a search request from a user having a user profile with user profile data;
defining a search domain based on the search request and the user profile data, wherein the search domain is a predefined collection of related searchable resources and includes the domain and content of a search web page; and
conducting a content search within the search domain.

2. The method of claim 1 further comprising:
generating a search results page based on search results of the content search.

3. The method of claim 1 further comprising:
prior to receipt of the search request, recognizing the user to determine the user profile, wherein the user profile data includes data entered by the user when registering with a search engine.

4. The method of claim 3, wherein the user registers with a web portal having the search engine available thereon.

5. The method of claim 1 further comprising:
logging the search request data in an aggregate data database;
determining additional users having related user profile data; and
storing search request data for the additional users in the aggregate data database.

6. The method of claim 1 further comprising:
aggregating the data in the aggregate data database;
recognizing profile commonalities between the users; and
recognizing search request commonalities between the users.

7. The method of claim 6 further comprising:
generating a notification based on the search request commonalities; and
notifying the user, based on the user profile data.

8. The method of claim 7, wherein the data relates to vehicle information, the method further comprising:
recognizing common vehicle-based search terms; and
aggregating the common vehicle-based search terms for users having related vehicle information as determined by the user profile data.

9. The method of claim 7, wherein the data relates to medical information, the method further comprising:
recognizing common medical-based search terms; and
aggregating the common medical-based search terms for user having related medical information as determined by the user profile data.

10. Computer readable media comprising program code that when executed by a processor causes the execution of a method for vertical searching, the computer readable media comprising:
program code for receiving a search request from a user having a user profile with user profile data;
program code for defining a search domain based on the search request and the user profile data, wherein the search domain is a predefined collection of related searchable resources and includes the domain and content of a search web page; and
program code for conducting a content search within the search domain.

11. The computer readable media of claim 10 further comprising:
program code for generating a search results page based on search results of the content search.

12. The computer readable media of claim 10 further comprising:
program code for recognizing the user to determine the user profile prior to receipt of the search request, wherein the user profile data includes data entered by the user when registering with a search engine.

13. The computer readable media of claim 12, program code for the user registering with a web portal having the search engine available thereon.

14. The computer readable media of claim 10 further comprising:
program code for logging the search request data in an aggregate data database;
program code for determining additional users having related user profile data; and
program code for storing search request data for the additional users in the aggregate data database.

15. The computer readable media of claim 10 further comprising:
program code for aggregating the data in the aggregate data database;
program code for recognizing profile commonalities between the users; and
program code for recognizing search request commonalities between the users.

16. The computer readable media of claim 15 further comprising:
program code for generating a notification based on the search request commonalities; and
program code for notifying the user, based on the user profile data.

17. The computer readable media of claim 16, wherein the data relates to vehicle information, the method further comprising:
program code for recognizing common vehicle-based search terms; and
program code for aggregating the common vehicle-based search terms for users having related vehicle information as determined by the user profile data.

18. The computer readable media of claim 16, wherein the data relates to medical information, the method further comprising:
program code for recognizing common medical-based search terms; and
program code for aggregating the common medical-based search terms for user having related medical information as determined by the user profile data.
